(19) United States
(12) Patent Application Publication
(10) Pub. No.: US 2024/0389962 A1
(43) Pub. Date: Nov. 28, 2024

(54) RADIOGRAPHIC IMAGING APPARATUS AND RADIOGRAPHIC IMAGING METHOD

(71) Applicant: DRTECH CORP, Gyeonggi-do (KR)

(72) Inventors: Choul Woo SHIN, Seongnam-Si, Gyeonggi-Do (KR); Won Taek SEO, Seongnam-si, Gyeonggi-Do (KR); Mi Jung JO, Suwon-si, Gyeonggi-Do (KR)

(21) Appl. No.: 17/921,348

(22) PCT Filed: Dec. 9, 2021
(86) PCT No.: PCT/KR2021/018647

§ 371 Date: Oct. 25, 2022

(30) Foreign Application Priority Data
Jun. 29, 2021 (KR) 10-2021-0084682

(51) Int. Cl. A61B 6/50; A61B 6/00; A61B 6/02

(52) U.S. Cl. CPC A61B 6/502; A61B 6/544; A61B 6/025

(57) ABSTRACT

The present inventive concept relates to a radiographic imaging apparatus and a radiographic imaging method for determining a radiographic mode according to a type of a subject's breast. The radiographic imaging apparatus may comprise a radiation source; a radiation detector; a type determination unit configured to determine the type of the breast using a radiation transmission value for the breast obtained from the radiation detector; and an imaging mode determination unit configured to determine the radiographic mode for the breast according to the type of the breast.

### TECHNICAL FIELD

The present disclosure relates to a radiographic imaging apparatus and a radiographic imaging method, and more particularly, to a radiographic imaging apparatus and a radiographic imaging method for determining a radiographic mode according to a type of a subject's breast.

### BACKGROUND

A digital-based imaging technology strongly reflects the clinical environment's demand for early diagnosis of disease based on the excellent diagnostic ability of digital imaging. In this regard, Digital Mammography, a breast-only radiographic technology to detect lesions and micro calcifications has been introduced for detection and early diagnosis of breast cancer. It employs a biological tissue contrast capability intrinsic to radiation (e.g., X-ray, etc.) to exhibit a high-resolution image of breast's internal structure as an object of radiographic imaging.

In general, a mammography system is a radiographic imaging apparatus used for early diagnosis of breast cancer, which transmits a certain amount of radiation to a subject's breast and detect the amount of transmitted radiation in a radiation detector to acquire two-dimensional images. Recently, Digital Breast Tomosynthesis (DBT) has been proposed, which is a breast cancer diagnosis technology using three-dimensional images to improve various limitations of the conventional breast cancer diagnosis technology using two-dimensional images.

It has been reported that 2D Mammography can reduce breast cancer mortality by about 20-30% in 40-74 years old women having an average risk in randomized controlled studies. The 2D Mammography is currently a standard method for breast cancer screening, but it has a limitation that it is difficult to detect breast cancer in women with dense breast tissues.

As younger women or women with dense breast are less sensitive than postmenopausal women or women with fatty breast, it may be more likely that breast cancer lesions are hidden by breast parenchyma, or the lesions are not recognized (false-negative diagnosis). Thus, in addition to 2D Mammography, the need for an auxiliary breast screening test is emerging, and this trend has been reflected in the US breast density notification law.

Digital Breast Tomosynthesis (DBT) acquires cross-sections from multiple angles and combines them to provide a single 3D breast image. Thus, this new screening method is expected to solve the problem of 2D Mammography by reducing the overlap of breast tissues.

The DBT was developed by modifying the conventional 2D Mammography to obtain 3D data on breast. In the field of breast imaging, DBT was first introduced in the 1990s and approved by the FDA in 2011. Many studies have been reported on its clinical use to date, and DBT is increasingly widely used over the world.

The DBT is a technology developed to overcome the disadvantage of 2D Mammography that the entire mammary gland tissue is superimposed on one image, and thus the sensitivity and specificity for breast cancer detection are lowered. In the conventional 2D Mammography, upper and lower or internal and external oblique images obtained from any one direction exhibit three-dimensional breast at a two-dimensional level, whereas DBT irradiates an X-ray source from various angles within a limited range to breast in a compressed state, and acquires multiple projection images from each cross-section in a digital radiation detector.

In DBT, if a radiation tube is moved within the range of 50° and each exposure is done every 2°, a total of 25 individual projection images will be acquired. These projection images are reconstructed using an algorithm similar to computed tomography or magnetic resonance imaging, whereby providing a 3D image. An image reading surgeon can visually evaluate breast by adjusting intervals between these reconstructed segments to 0.5 to 1 mm, and can see volume data for the entire breast. For example, if a 5 cm thick breast is reconstructed at 1 mm intervals, a total of 50 images are produced, and if the same breast is reconstructed to a 0.5 mm thickness, 100 images are produced. Alternatively, if it is reconstructed to a 10 mm plate thickness by the maximum intensity projection image, 5 images are produced.

Several DBT manufacturers are developing DBTs by applying different methods, and each manufacturer has different advantages and disadvantages. Depending on each manufacturer, an angle at which a radiation tube moves (usually 15-50°), the number of radiation exposures (usually 9-25 times), whether a radiation detector moves (individual rotation/tilting), the use of continuous or intermittent exposure (Step and shoot and/or Continuou), exposure parameters, effective pixel size, reconstruction method, etc. are different.

In the field of DBT, it is currently possible to generate a 3D tomography image using an existing 2D mammography image as well as a plurality of 2D slice images, and also to produce an additional 2D image by merging or synthesizing such 3D tomography images.

The above-mentioned DBT has increased sensitivity during screening, and exhibits the removed or reduced overlap of breast tissues, resulting in lowering a false negative rate due to normal breast tissues covering breast cancer, and a false positive rate caused by the overlapping shading due to normal breast tissues overlapped. Also, DBT can reduce a recall rate, which is particularly useful in women with dense breast tissues.

Theoretically, the reading through thin segments would improve the detection rate of lesions that may be obscured by tissues overlapped, and DBT is primarily useful for discovering and characterizing masses not accompanied by calcification, asymmetric shading, and structural distortions. That is, DBT has an advantage that smaller sized lesions can be detected through three-dimensional images obtained from a series of exposures within a limited angle, so that the location of lesions can be easily identified, and the characterization of lesions such as benign and malignant can be improved. In DBT, sensitivity as well as specificity can be improved such that malignant lesions appear more malignant and benign lesions appear more benign.

The conventional 2D mammography has reduced sensitivity to dense breast, and DBT can dramatically improve the accuracy of the diagnosis. The addition of DBT to 2D mammography resulted in a dramatic improvement in the diagnosis accuracy across almost all breast densities, particularly increase more than twice in dense breast than in fatty breast.

Overall, it can be appreciated that DBT has improved diagnostic ability to detect breast cancer in women with dense breast. Also, as DBT performs the reading through thin segments, it has a great advantage that edge boundaries that may be obscured by tissues overlapped in lesions detected can be more accurately evaluated.

In addition, DBT, which provides three-dimensional images, can detect smaller lesions compared to the conventional mammography, and help in determining the location of lesions. If a lesion in breast is visible only in upper and lower, or internal and external oblique images, the location of the lesion can be determined using DBT. The DBT may be read after adjusting intervals between segments at a workstation, so that the size or distribution of lesions in breast can be identified.

A majority of Asians below the age of 40 have dense breast, but there is a limitation that the sensitivity and specificity of diagnosis are still low in such dense breast. That is, as the size and tissue density of breasts are different depending on women's age and race, an imaging angle (mode) for acquiring DBT images must be very accurate according to the type of a subject's breast.

In DBT imaging, a narrow range of imaging angle (narrow angle) facilitates the detection of micro-calcifications, a wide range of imaging angle (wide angle) facilitates the detection of masses, and a medium range of imaging angle facilitates the detection of structural distortion. At a narrow angle, a scan time is shortened and a radiation dose is low, but the performance of images in horizontal direction is lowered. On the contrary, at a wide angle, the performance of images in horizontal direction is improved, but more projection images are required to obtain an image, so a scan time is prolonged and a dose exposed to a subject is increased. Many manufacturers have developed DBTs by applying different methods.

In addition, since parameters such as an area ratio may be varied depending on a breast compression method or a positioning in DBT imaging, there is a problem that it is fundamentally difficult to take a picture at an angle (mode) optimized for the type of a subject's breast.

In summary, it is necessary to accurately identify the type of a subject's breast and take a picture at an imaging mode optimized for the identified breast type such that unnecessary radiation doses exposed to a subject during DBT imaging is reduced while acquiring good quality images. However, there is still no manufacturer that provides such an imaging mode.

### REFERENCE

Patent Document 1—Korean Patent No. 10-2179319

### Technical Task

An object of the present inventive concept is to provide a radiographic imaging apparatus and a radiographic imaging method to appropriately determine a radiographic mode such as an imaging angle according to a type of a subject's breast.

### Means for Solving the Task

According to an embodiment of the present inventive concept, a radiographic imaging apparatus may comprise a radiation source configured to irradiate radiation to a subject's breast and to be rotatable around the breast; a radiation detector configured to detect radiation that has passed through the breast; a type determination unit configured to determine a type of the breast using a radiation transmission value for the breast obtained from the radiation detector; and an imaging mode determination unit configured to determine a radiographic mode for the breast according to the type of the breast.

The imaging mode determination unit may comprise a rotation angle determination unit configured to determine an angle range in which the radiation source rotates according to the type of the breast.

The type determination unit may comprise at least one of a thickness measurement unit configured to measure a thickness of the breast; an area measurement unit configured to measure a plane area of the breast; and a density information acquisition unit configured to acquire a relative density information on the breast, and may determine the type of the breast using at least one of the thickness, the plane area and the relative density information of the breast.

The rotation angle determination unit may determine the angle range in inverse proportion to a relative density of the breast.

The density information acquisition unit may acquire the relative density information on the breast using the radiation transmission value obtained by a pre-shot.

A relative density of the breast may be inversely proportional to the radiation transmission value.

The pre-shot may be performed at an irradiation dose determined according to the thickness of the breast.

The imaging mode determination unit may further comprise an irradiation dose determination unit configured to determine an irradiation dose to the breast according to at least one of the thickness, the plane area and the relative density information of the breast.

The imaging mode determination unit may further comprise a resolution determination unit configured to determine a radiographic image resolution according to at least one of the irradiation dose and the angle range.

The imaging mode determination unit may further comprise an imaging frequency determination unit configured to determine the number of radiographic imaging runs according to at least one of the irradiation dose and the angle range.

According to further embodiment of the present inventive concept, a radiographic imaging method may comprise the steps of positioning a subject's breast between a radiation detector and a compression unit, for radiography; determining a type of the breast; and determining a radiographic mode for the breast according to the type of the breast.

The determining the radiographic mode may comprise determining an angle range in which a radiation source rotates around the breast according to the type of the breast.

The determining the type of the breast may determine the type of the breast using at least one of a thickness, a plane area and a relative density information of the breast.

The determining the angle range may determine the angle range in inverse proportion to a relative density of the breast.

The radiographic imaging method may further comprise the step of performing a pre-shot on the breast, and the determining the type of the breast may comprise acquiring the relative density information of the breast by obtaining a detection value of the radiation detector detected in the performing the pre-shot.

The performing the pre-shot may be performed at an irradiation dose determined according to the thickness of the breast.

The acquiring the relative density information of the breast may calculate a relative density of the breast according to an equation inversely proportional to the detection value of the radiation detector.

The determining the radiographic mode may further comprise determining an irradiation dose to the breast according to at least one of the thickness, the plane area and the relative density information of the breast.

The determining the radiographic mode may further comprise determining a radiographic image resolution according to at least one of the irradiation dose and the angle range.

The determining the radiographic mode may further comprise determining the number of radiographic imaging runs according to at least one of the irradiation dose and the angle range.

### The Effect of the Invention

A radiographic imaging apparatus according to an embodiment of the present inventive concept determines the type of a subject's breast in a type determination unit, so that a radiographic mode such as an imaging angle can be suitably determined according to the type of the breast. Thus, the accuracy of lesion detection can be improved while minimizing the exposure dose to the subject.

As such, minute lesions that may develop into tumors can be perfectly detected at an early stage, which has an effect of minimizing the incidence of breast cancer. Also, by determining an angle range in which a radiation source rotates around the breast according to the type of the breast, it is possible to effectively detect micro-calcifications in dense breast, and also masses in fatty breast.

In acquiring the relative density information of the breast, the relative density information is acquired by performing a pre-shot on the breast at an irradiation dose determined according to the thickness of the breast. Thereby, the accuracy of the relative density calculation can be improved, as well as the relative density information thus acquired on the breast is used to determines a radiographic mode such as an angle range in which a radiation source rotates and to perform the radiographic imaging, resulting in a high-quality diagnostic image on the breast. Thus, there is an effect that can solve various problems in the field of breast cancer diagnosis.

Additionally, in determining a radiographic mode (e.g., the radiation source rotation angle range), the mode is determined according to the type of the breast which is identified using at least one of the thickness, the plane area and the relative density information of the breast. Thereby, the radiographic imaging can be performed in a radiographic mode such as an angle range in which a radiation source rotates which is optimized for the type of the breast, as well as defects (or noises) such as cutting artifacts generated during image reconstruction for a three-dimensional (3D) tomosynthesis image can be minimized. As a result, the lesion detectability and accuracy can be maximized and the reliability of medical examination and diagnosis can be improved.

### BRIEF DESCRIPTION OF THE DRAWINGS

FIG. 1 is a cross-sectional view schematically showing a radiographic imaging apparatus according to an embodiment of the present inventive concept.

FIG. 2 is a schematic diagram showing a radiographic imaging apparatus according to an embodiment of the present inventive concept.

FIG. 3 is a block diagram showing a radiographic imaging apparatus according to an embodiment of the present inventive concept.

FIG. 4 is a conceptual diagram for explaining a rotation angle range of a radiation source according to an embodiment of the present inventive concept.

FIG. 5 is a conceptual diagram for explaining the determination of an imaging angle in which the area of the breast is reflected according to an embodiment of the present inventive concept.

FIG. 6 is a flowchart of a radiographic imaging method according to further embodiment of the present inventive concept.

### DETAILED DESCRIPTION

Hereinafter, the embodiments of the present inventive concept will be described in more detail below with reference to the accompanying drawings. However, the present inventive concept is not limited to these embodiments described below, and may be implemented in various different forms. Only these embodiments are provided to complete the disclosure of the present inventive concept, and to fully explain the present inventive concept to those of ordinary skill in the art. Throughout the description, the same reference numerals are assigned to the same elements, and the size of the drawings may be partially exaggerated in order to accurately describe the embodiments of the present inventive concept. In the drawings, like reference numerals refer to like elements.

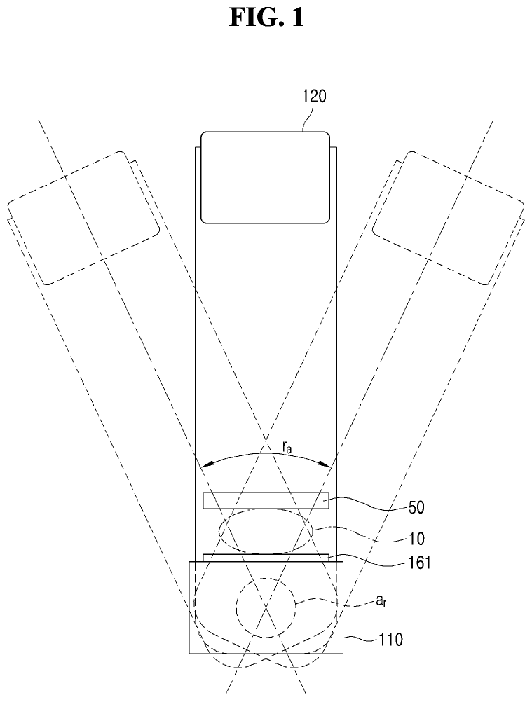

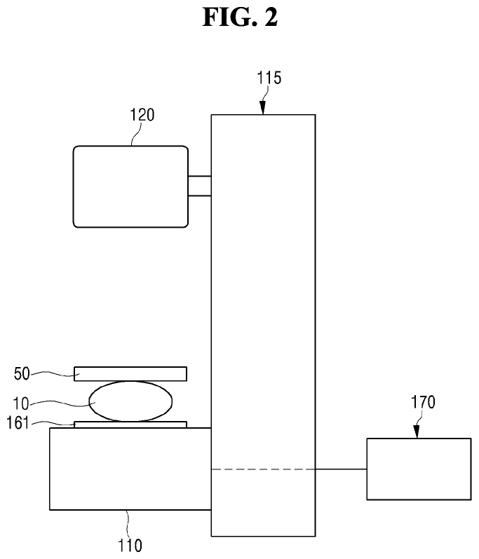

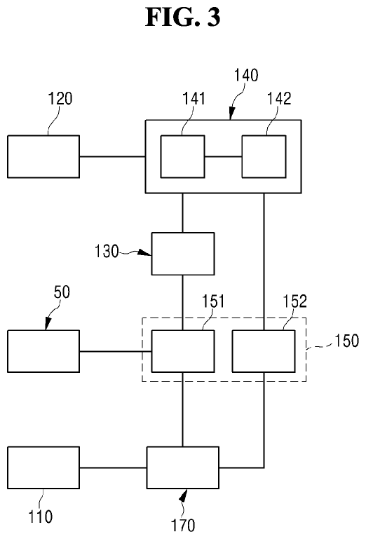

FIG. 1 is a cross-sectional view schematically showing a radiographic imaging apparatus according to an embodiment of the present inventive concept, FIG. 2 is a schematic diagram showing a radiographic imaging apparatus according to an embodiment of the present inventive concept, and FIG. 3 is a block diagram showing a radiographic imaging apparatus according to an embodiment of the present inventive concept.

Referring to FIG. 1 to FIG. 3, a radiographic imaging apparatus 100 according to an embodiment of the present inventive concept may comprise a radiation source 120 configured to irradiate radiation to a subject's breast 10 and to be rotatable around the breast 10; a radiation detector 110 configured to detect radiation that has passed through the breast; a type determination unit 130, 150 configured to determine a type of the breast 10 using a radiation transmission value for the breast 10 obtained from the radiation detector 110; and an imaging mode determination unit 140 configured to determine a radiographic mode for the breast 10 according to the type of the breast 10.

The radiation detector 110 may detect radiation (e.g., X-ray, etc.). It may detect radiation that has passed through the subject's breast 10 to acquire image information. For example, the radiation detector 110 may have two surfaces facing each other, and may detect radiation irradiated (or incident) on any one of the two surfaces facing each other. In this case, the radiation detector 110 may be positioned to correspond to the radiation source 120, and may detect radiation that has passed through the breast 10. The radiation detector 110 may be a film radiation detector using a film that is photosensitized by radiation, and a computed radiography (CR) radiation detector in which a digital image is acquired using an image plate (IP) instead of an analog film, or a digital radiography (DR) radiation detector in which radiation is electrically detected by a semiconductor sensor or the like without a film to acquire image information.

The radiation source 120 may irradiate radiation to the breast 10 on the radiation detector 110. It may rotate around the breast 10, and may perform the radiographic imaging on the breast 10 from a plurality of (or various) angles. For example, the radiographic imaging apparatus 100 according to the present inventive concept may further comprise a rotation driving unit (not shown) to rotate the radiation source 120 around the breast 10. The rotation driving unit (not shown) may rotate the radiation source 120 around the breast 10; it may rotate the radiation source 120 about a rotation axis (ar) inside or outside the radiation detector 110 on an extension of a line connecting the radiation source 120 and the radiation detector 110, or it may rotate the radiation source 120 about the radiation detector 110 or the breast 10. The rotation driving unit (not shown) may rotate the radiation source 120 independently in a state that the radiation detector 110 is fixed, or may rotate the radiation source 120 and the radiation detector 110 together (or simultaneously) in a state that the radiation source 120 and the radiation detector 110 face each other.

In other words, the radiation source 120 may be rotatable along the periphery of the breast 10 by the rotation driving unit (not shown); it may rotate around the breast 10 about a rotation axis (ar) inside or outside the radiation detector 110, or it may rotate around the breast 10 about the radiation detector 110 or the breast 10. Also, the radiation source 120 may rotate independently from the radiation detector 110, or may rotate with the radiation detector 110 while facing each other.

For example, the radiation source 120 may be an X-ray tube, and also may irradiate radiation by limiting a radiation irradiation area through a collimator.

The radiation source 120 and the radiation detector 110 may be connected to each other by a connection frame 115. They may be aligned and connected to each other. Also, they may be aligned to each other by positioning the radiation source 120 and the radiation detector 110 at opposite angles (e.g., 90° and 270°) while being connected to each other by the connection frame 115.

The type determination unit 130, 150 may determine the type of the breast 10 using a radiation transmission value for the breast 10 obtained from the radiation detector 110, and the determined type of the breast 10 may be transmitted to the imaging mode determination unit 140. The radiation transmittance value may comprise data consisting of a single value or a plurality of values, and the type of the breast 10 may comprise a size and/or (relative) density of the breast 10.

The size and tissue density of breasts may be different depending on the subject's age, race, etc. Conventionally, Digital Breast Tomosynthesis (DBT) was performed at the same angle for all subjects within an imaging angle fixed for each equipment, which resulted in a problem that the quality of three-dimensional (3D) tomography images was varied due to differences between subjects in the size and tissue density of the breast 10. Due to this problem, micro-calcifications could not be detected in the breast 10 with dense tissues in a wide imaging angle, or masses could not be detected in the breast 10 with fatty tissues in a narrow imaging angle. As a result, the lesion detectability and accuracy, as well as the reliability of medical examination and diagnosis are lowered. On the contrary, according to the present inventive concept, since the type of the breast 10 may be determined and the result may be reflected in the radiographic imaging of the breast 10, it is possible to produce (or acquire) a three-dimensional (3D) tomography image with excellent quality regardless of (or without influence) of the type of the breast 10 such as size and tissue density (or relative density).

The imaging mode determination unit 140 may determine a radiographic mode for the breast 10 according to the type of the breast 10, and also an irradiation dose and a rotation angle of the radiation source 120 may be determined according to the type of the breast 10 determined in the type determination unit 130, 150. By using the imaging mode determination unit 140, it is possible to determine a radiographic mode such as a rotation angle and an irradiation dose of the radiation source 120 suitable for the breast 10 according to a relative density of the breast 10. Thus, the radiographic imaging suitable for the type of the breast 10 can be performed, and the accuracy of lesion detection can be improved while minimizing the exposure dose to the subject.

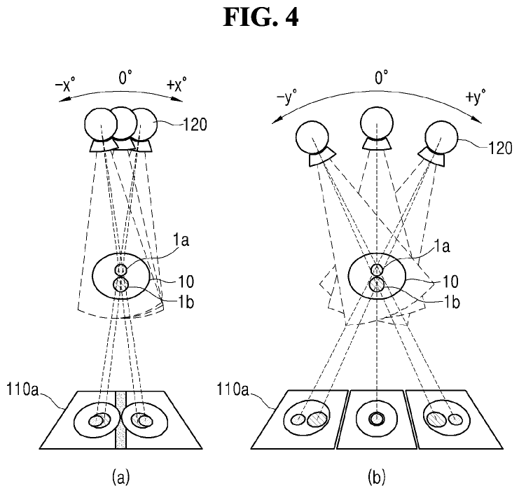

FIG. 4 is a conceptual diagram for explaining a rotation angle range of a radiation source according to an embodiment of the present inventive concept; (a) of FIG. 4 shows a narrow rotation angle range, and (b) of FIG. 4 shows a wide rotation angle range.

Referring to FIG. 4, in a narrow rotation angle range (rₐ) as shown in (a) of FIG. 4, it may be difficult to separate a projection plane 110a between each object 1a and 1b adjacent in the vertical direction. On the contrary, in a wide rotation angle range (rₐ) as shown in (b) of FIG. 4, it may be easy to separate a projection plane 110a between each object 1a and 1b adjacent in the vertical direction. As the rotation angle range (rₐ) is widened, the image performance in horizontal direction can be improved, but there are disadvantages that more projection images are required to acquire a radiographic image and a scan time is prolonged. As the rotation angle range (rₐ) becomes narrower, a scan time is shortened and an exposure dose can be reduced, but there is a disadvantage that the image performance in horizontal direction is deteriorated. Here, the objects 1a and 1b may be any lesion such as tumor. It may be easy to detect micro-calcifications in the narrow rotation angle range (rₐ), whereas it may be easy to detect masses (or lumps) in the wide rotation angle range (rₐ).

The imaging mode determination unit 140 may comprise a rotation angle determination unit 141 to determine an angle range (rₐ) at which the radiation source 120 rotates according to the type of the breast 10. The rotation angle determination unit 141 may determine an angle range (rₐ) at which the radiation source 120 rotates according to the type of the breast 10, and may determine the angle range (rₐ) suitable for the breast 10 according to the type of the breast 10.

Herein, the type determination unit 130, 150 may comprise at least one of a thickness measurement unit 151 configured to measure a thickness of the breast 10; an area measurement unit 152 configured to measure a plane area of the breast 10; and a density information acquisition unit 130 configured to acquire a relative density information on the breast 10.

The thickness measurement unit 151 is configured to measure the thickness of the breast 10 and reflect the measured thickness of the breast 10 to the type (information) of the breast 10. For example, the thickness measurement unit 151 may detect the thickness (value) of the breast 10, and may acquire the thickness information of the breast 10 by various methods capable of detecting the thickness of the breast 10.

The radiographic imaging apparatus 100 according to the present inventive concept may further comprise a compression unit 50 to press the breast 10, provided on the radiation detector 110.

The compression unit 50 may be provided on the radiation detector 110 to press the breast 10 on the radiation detector 110. The thickness measurement unit 151 may detect (or acquire) the thickness of the breast 10 using a distance between the compression unit 50 and the radiation detector 110. The compression unit 50 may be consisted of only a compression panel to press the breast 10 between the compression panel and the radiation detector 110, or may be consisted of a compression panel and a support panel to press the breast 10 between the compression panel and the support panel.

Also, the thickness measurement unit 151 may be configured to detect a position of the compression panel using a sensor and detect the thickness of the breast 10 based on a value received from the sensor. Alternatively, the thickness measurement unit 151 may be configured to monitor the operation of a driving motor (not shown) for moving the compression panel and detect the thickness of the breast 10 based on the monitoring result. Here, the thickness of the breast 10 may be detected based on the position (e.g., the rotation angle of rotor) of the driving motor (not shown).

Specifically, the thickness measurement unit 151 may comprise a distance measurement sensor (not shown), which is installed on either the compression panel or the supporting panel, or both the compression panel and the supporting panel. The thickness of the breast 10 may be also measured by measuring a distance between the compression panel and the support panel using the distance measurement sensor. The distance measurement sensor may be an infrared, ultrasonic, or laser sensor. Also, the thickness measurement unit 151 may comprise a gear (not shown) which rotates to adjust a height of the compression panel and is installed on a support on which the compression panel and the support panel are supported. The thickness of the breast 10 may be also measured by measuring the revolutions of the gear.

The area measurement unit 152 is configured to measure the plane area (e.g., horizontal cross-sectional area) of the breast 10 and reflect the measured plane area of the breast 10 to the type (information) of the breast 10. For example, the area measurement unit 152 may calculate the plane area (value) of the breast 10, and may transmit the calculated plane area of the breast 10 to the imaging mode determination unit 140. For example, the calculated plane area of the breast 10 may be transmitted to the rotation angle determination unit 141 and be reflected to determine the angle range (rₐ) or to determine the irradiation angle of the radiation source 120. Here, the area measurement unit 152 may calculate the area of the breast 10 using a radiographic image obtained from a pre-shot on the breast 10.

The area of the breast 10 may be calculated using a radiographic image obtained from a pre-shot on the breast 10. The radiographic image obtained from the pre-shot on the breast 10 may be a pre-shot image. The outline of the breast 10 may be identified (or acquired) along the light and dark distribution of the radiographic image obtained from the pre-shot (i.e., the pre-shot image), and the area of the breast 10 can be calculated as the area of the breast 10 within the radiographic image obtained from the pre-shot by using a ratio of the area of the radiographic image obtained from the pre-shot and the detection area of the radiation detector 110. The area of the breast 10 within the radiographic image obtained from the pre-shot may be calculated (or obtained) by the number of pixels. For example, if a ratio of the area of the radiographic image obtained from the pre-shot and the detection area of the radiation detector 110 is 1:10, the area of the breast 10 can be calculated by multiplying the area of the breast 10 within the radiographic image obtained from the pre-shot by 10 times.

Additionally, the thickness measurement unit 151 and the area measurement unit 152 may form a size information acquisition unit 150 to acquire the size information of the breast 10. The size information acquisition unit 150 may acquire the size information of the breast 10, and may transmit the size information acquired on the breast 10 to the imaging mode determination unit 140. For example, the size information of the breast 10 may be transmitted to the rotation angle determination unit 141 and be reflected to determine the angle range (rₐ).

The density information acquisition unit 130 may acquire the relative density information on the breast 10, and may transmit the relative density information acquired on the breast 10 to the imaging mode determination unit 140.

The type determination unit 130, 150 may determine the type of the breast 10 using at least one of the thickness, the plane area and the relative density information of the breast 10. The type determination unit 130, 150 may determine the type of the breast 10 by at least one or a combination of two or more of the thickness, the plane area and the relative density information of the breast 10. For example, the relative density of the breast 10 may be divided into a dense/slight dense/medium/fatty type; the thickness of the breast 10 may be divided into thick/medium/thin; and the plane area of the breast 10 may be divided into narrow/medium/wide. By combining these various types, combinational n types of the breast 10 may be detected, and in particular, n types including the combination of dense/thick/narrow may be detected. Meanwhile, if the type of the breast 10 is the first type of dense/thick/narrow, a narrow radiographic mode may be used wherein a rotation angle range is within 6.5 to 8.5° (about 7.5°), and an irradiation dose for the narrow mode may be determined.

For example, the rotation angle determination unit 141 may determine the angle range (rₐ) at which the radiation source 120 rotates around the breast 10 according to the (relative) density of the breast 10, and hence micro-calcifications can be effectively detected in the dense breast 10, and masses can be effectively detected in the fatty breast 10. Thereby, minute lesions that may develop into tumors can be perfectly detected at an early stage, which can minimize the incidence of breast cancer.

Also, the rotation angle determination unit 141 may determine the angle range (rₐ) in inverse proportion to the relative density of the breast 10. As there are few factors to reduce (or diminish) radiation from tissues other than lesions in the fatty breast 10 having low density (e.g., relative density less than 25%), micro-calcifications can be easily detected by two-dimensional (2D) images alone. However, masses cannot be detected by two-dimensional (2D) images alone. For the detection of masses, it is necessary to generate three-dimensional (3D) tomography images by performing radiographic imaging at a plurality of angles while rotating the radiation source 120 around the fatty breast 10.

Considering that the wider angle range (rₐ) facilitates the mass detection, the radiographic imaging may be performed while rotating the radiation source 120 within a wide rotation angle range (rₐ) for the purpose of detecting masses. In this case, a high-quality (e.g., high-brightness) radiographic image can be also acquired from the fatty breast 10 at a low radiation dose. Thus, the exposure dose may not be a problem even if the irradiation is done for a long scan time at a low radiation dose in a wide rotation angle range (rₐ).

On the contrary, in the dense breast 10 having high density (e.g., relative density greater than 75%), the mass detection is difficult, and the detection of micro-calcifications is more difficult. Since the transmitted radiation is significantly diminished (or reduced), the radiation should be irradiated at a relatively high dose than in low density breast to acquire a radiographic image having a quality level for effective examination. That is, in the dense breast 10, the radiographic imaging may be performed while rotating the radiation source 120 within a narrow rotation angle range (rₐ). In this case, masses in the dense breast 10 can be detected by a radiographic image(s) taken at a high radiation dose and two or more imaging angles. Thus, for the dense breast 10, the angle range (rₐ) may be determined as a narrow rotation angle range (rₐ) focusing on the detection of micro-calcifications which are very difficult to detect.

Also, since the dense breast 10 is irradiated at a relatively high radiation dose, it may be advantageous to rotate the radiation source 120 within a narrow rotation angle range (rₐ) in terms of the exposure dose.

For example, the relative density of the breast 10 may be classified into four types. From high to low relative density: the order may be 1) high density dense breast 10 (e.g., relative density greater than 75%), 2) medium density breast 10 (e.g., relative density 51-75%), 3) breast 10 with mammary glands scattered (e.g., relative density 25-50%) and 4) fatty breast 10 (e.g., relative density less than 25%). The rotation angle range (rₐ) may be a narrow rotation angle range (rₐ) of 6.5 to 8.5° (about 7.5°) for the dense breast 10; a little narrow rotation angle range (rₐ) of 8.5 to 10.5° (about 9.5°) for the medium density breast 10; a normal rotation angle range (rₐ) of 11.5 to 14.5° (about 12.5°) for the breast 10 with mammary glands scattered; and a wide rotation angle range (rₐ) of 14.5 to 25° (about 25°).

As such, the angle range (rₐ) may be determined in inverse proportion to the relative density of the breast 10 in such a way that the higher the relative density of the breast 10, the narrower the rotation angle range (rₐ).

Also, considering that the irradiation dose should be raised (or increased) in proportion to the relative density of the breast 10 to acquire a high-quality radiographic image, the higher the irradiation dose in the breast 10 having high relative density, the angle range (rₐ) may be inversely narrowed in terms of the exposure dose. For example, the rotation angle determination unit 141 may determine the angle range (rₐ) according to the relationship between the rotation angle range (rₐ) and the relative density of the breast 10 using a calculation formula or a lookup table. That is, the angle range (rₐ) may be determined (or set) by preparing a table in which the information on the rotation angle range (rₐ) according to the relative density of the breast 10 is written, and then selecting in the table the rotation angle range (rₐ) corresponding to said information on the relative density of the breast 10 (i.e., the relative density information on the breast).

The density information obtaining unit 130 may acquire the relative density information on the breast 10 by using a radiation transmission value obtained by a pre-shot. That is, the density information obtaining unit 130 may acquire the relative density information on the breast 10 by using the detection value of the radiation detector 110 obtained from the pre-shot of the breast 10. Here, the pre-shot may be a shot performed prior to a main shot to confirm the tissue characteristics of the breast 10 and to set imaging conditions required for the main shot (i.e., radiographic mode as described above) according to the confirmed tissue characteristics. The main shot may be a shot performed according to the radiographic mode determined in the imaging mode determination unit 140.

For example, the radiographic imaging apparatus 100 according to the present inventive concept may perform Auto Exposure Control (AEC), such that the radiation exposure can be automatically controlled by setting imaging conditions depending on the type (size, density, etc.) of the breast 10. In this case, a pre-shot image may be used to identify the type of the breast 10, and the pre-shot image may be produced (or acquired) in an imaging processing unit 170 by performing the pre-shot.

As such, once the pre-shot image is acquired by the pre-shot, a detection value (e.g., a pixel value of each pixel of the radiation detector) may be detected (or acquired) in the radiation detector 110, and the relative density of the breast 10 can be calculated according to the detection value of the radiation detector 110. For example, the detection value of the radiation detector 110 may be pixel values (e.g., charge amounts) read-out from each pixel of the radiation detector 110. Only the pixel value of pixel (s) corresponding to the breast 10 (or the pixel value of the pixel (s) onto which the image of the breast is projected) may be used, or the average or median value of the pixel value (s) (e.g., values excluding maximum and minimum values) may be used.

Here, the relative density of the breast 10 may be inversely proportional to the radiation transmission value. That is, the relative density of the breast 10 may be inversely proportional to the detection value of the radiation detector 110 via the pre-shot. When the relative density of the breast 10 is high, the radiation is largely diminished while passing through the breast 10, so that the detection value of the radiation detector 110 may be low. When the relative density of the breast 10 is low, the radiation may be less diminished while passing through the breast 10, so that the detection value of the radiation detector 110 may be high. Based on this, when the detection value of the radiation detector 110 is low, it may be determined that the relative density of the breast 10 is high, while when the detection value of the radiation detector 110 is high, it may be determined that the relative density of breast 10 is low. Again, the relative density of the breast 10 may be inversely proportional to the detection value of the radiation detector 110. Taking this into account, an equation that is inversely proportional to the detection values of the radiation detector 110 may be produced, and the relative density of the breast 10 may be calculated using the equation.

The pre-shot may be performed at an irradiation dose that is determined depending on the thickness (value) of the breast 10. The thickness of the breast 10 may be measured (or detected) by the thickness measurement unit 151. The diminished amount of radiation which passes through the breast 10 may vary depending on the thickness of the breast 10. Thus, the diminished amount of radiation may large (or increase) as the thickness of the breast 10 increases. For this reason, even when the pre-shot is performed at the same irradiation dose for all types for the breast 10, the radiation passing through the breast 10 is diminished according to the thickness of the breast 10. Overall, the detection value of the radiation detector 110 is affected not only the relative density of the breast 10 but also by the thickness, so it is impossible to accurately calculate the relative density of the breast 10.

However, according to the present inventive concept, when the pre-shot is performed at an irradiation dose determined depending on the thickness of the breast 10, a difference in radiation diminishment between the breast's densities at the same thicknesses can be calculated, and thus it is possible to accurately calculate the relative density of the breast 10.

As such, to acquire the relative density information on the breast 10, the pre-shot may be performed on the breast 10 at an irradiation dose determined depending on the thickness of the breast 10, thereby improving the accuracy of the relative density calculation. Also, the radiographic mode can be determined according to the relative density information acquired on the breast 10, and the radiographic imaging can be performed according to the determined radiographic mode. Thereby, a diagnostic image of the breast 10 with improved quality can be obtained, and various problems in the field of breast cancer diagnosis can be solved.

For example, the diminished amount of radiation is increased as the thickness of the breast 10 increases, so the radiation dose of the pre-shot can be determined in proportion to the thickness of the breast 10. The radiation dose of the pre-shot may be empirically determined according to the thickness of the breast 10.

The density information acquisition unit 130 may calculate the relative density of the breast 10 using a calculation formula or a lookup table that is determined for the thickness of the breast 10. The relative density of the breast 10 may be calculated according to the relationship between the detection value of the radiation detector 110 and the relative density of the breast 10 for the thickness of the breast 10. For example, the relative density of the breast 10 can be acquired (or calculated) by preparing a table in which the information on the relative density of the breast 10 according to the value detected from the radiation detector 110 for the thickness of the breast 10 is written, and then selecting in the table the relative density of the breast 10 corresponding to the information on the detection value of the radiation detector 110.

The rotation angle determination unit 141 may determine the angle range (rₐ) by (further) reflecting the size information of the breast 10. By reflecting (further) the size information of the breast 10 to determine the angle range (rₐ), the radiographic imaging may be performed on the breast 10 at the optimized rotation angle range (rₐ) for the type of the breast 10.

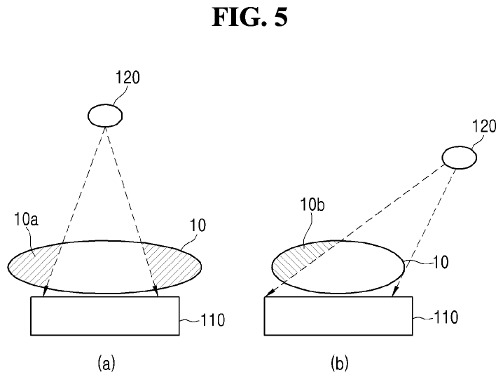

FIG. 5 is a conceptual diagram for explaining the determination of an imaging angle in which the area of the breast is reflected according to an embodiment of the present inventive concept; (a) of FIG. 5 shows the radiograph on the breast with a large area using a narrow rotation angle range, and (b) of FIG. 5 shows the radiograph on the breast with a small area using a wide rotation angle range.

Referring to FIG. 5, when the radiographic imaging is performed on the breast 10 with a large (plane) area using a narrow rotation angle range (rₐ) as shown in (a) of FIG. 5, a mass that may be on the outer side 10a of the breast 10 greater than the irradiation angle of the radiation source 120 may be missed. Also, as shown in (b) of FIG. 5, when the radiographic imaging is performed on the breast 10 with a small (plane) area using a wide rotation angle range (rₐ), a micro-calcification cluster that may be on the outer side 10b of the breast 10, which is a blind spot due to the shooting angle, may be missed.

The size information of the breast 10 may comprise an area (value) of the breast 10. If the angle range (rₐ) is determined without considering the area of the breast 10, the radiographic imaging may be performed on the breast 10 having large area using the narrow rotation angle range (rₐ), or the radiographic imaging may be performed on the breast 10 having small area using the wide rotation angle range (rₐ). In this case, as described above, a mass that may be on the outer side 10a of the breast 10 may be missed, or a micro-calcification cluster that may be on the outer side 10b of the breast 10 may be missed. To avoid this problem, the angle range (rₐ) in which the area of the breast 10 is reflected may be determined. The angle range (rₐ) may be proportional to the area of the breast 10.

As such, the rotation angle determination unit 141 may determine the angle range (rₐ) in which the area of the breast 10 is reflected.

The rotation angle determination unit 141 may determine the angle range (rₐ) by (further) reflecting the thickness of the breast 10. For example, the angle range (rₐ) may decrease as the thickness of the breast 10 increases, while the angle range (rₐ) may increase as the thickness of the breast 10 decreases. That is, it may be inversely proportional to the thickness of the breast 10. To image the entire breast 10 using the radiation source 120 having a fixed irradiation angle, the radiation source 120 should rotate around the breast 10. In this case, the angle range (rₐ) may be decreased as the thickness of the breast 10 increases. That is, as the thickness of the breast 10 is thicker, the upper end of the breast 10 can be closer to the radiation source 120, so that the entire breast 10 can be imaged even with a narrow angle range (rₐ). On the contrary, as the thickness is thinner, the upper end of the breast 10 moves away from the radiation source 120. Thus, the angle range (rₐ) should be widened such that the entire breast 10 can be imaged.

To this end, the rotation angle determination unit 141 may determine the angle range (rₐ) in which the thickness of the breast 10 is reflected to image the entire breast 10 using the radiation source 120 having a fixed irradiation angle. That is, when the radiation source 120 has a fixed irradiation angle, it may be prevented that the angle range is determined only by the relative density of the breast, thereby ensuring that the entire breast 10 is imaged.

According to the present inventive concept, the radiographic mode (e.g., the rotation angle range of the radiation source) may be determined according to the type of the breast 10 that is identified using at least one of the thickness, the (plane) area and the relative density information of the breast 10, and thus the radiographic imaging may be performed on the breast 10 in the optimized radiographic mode such as the optimized rotation angle range of the radiation source 120 for the type of the breast 10. Thus, defects (or noises) such as cutting artifacts generated during image reconstruction for a three-dimensional (3D) tomosynthesis image can be minimized. As a result, the lesion detectability and accuracy can be maximized and the reliability of medical examination and diagnosis can be improved.

The rotation angle determination unit 141 may determine the angle range (rₐ) by applying a weight to the relative density, thickness, and (plane) area of the breast 10. Since the relative density and thickness of the breast 10 are inversely proportional to the angle range (rₐ), and the plane area of the breast 10 is proportional to the angle range (rₐ), if a wight is not applied to the relative density, thickness and (plane) area of the breast 10, the rotation angle range (rₐ) may be determined to be uncertain such as a normal range or a slight narrow range regardless of the relative density of the breast 10. As a result, it is impossible to perform the radiographic imaging suitable for the type of the breast 10, as well as there may be problems that the exposure dose to the breast 10 is excessively increased, or it is difficult to accurately detect lesions using the produced (or acquired) radiographic images. Thus, a weight may be applied to the relative density, thickness, and (plane) area of the breast 10, and the rotation angle determination unit 141 may determine the rotation angle range (rₐ) with the weight applied to the relative density, thickness, and (plane) area of the breast 10.

For example, the largest weight may be applied to the relative density of the breast 10, and the thickness and (plane) area of the breast 10 may be weighted according to the overall size of the breast 10.

The radiographic mode determination unit 140 may further comprise an irradiation dose determination unit 142 to determine the irradiation dose to the breast 10 according to at least one of the thickness, the plane area and the relative density information of the breast 10. The irradiation dose determining unit 142 may determine the irradiation dose to the breast 10 according to at least one of the thickness, the plane area and the relative density information of the breast 10, and the optimal irradiation dose may be determined by varying exposure parameters in proportion to or inverse proportion to at least one or any combination (combined data) of two or more of the relative density, thickness and plane area of the breast 10. For example, the irradiation dose determination unit 142 may determine the irradiation dose to the breast 10 in proportion to the relative density of the breast 10, and the irradiation dose of the main shot may be determined. The irradiation dose determination unit 142 may also determine the irradiation dose to the breast 10 in proportion to the thickness of the breast 10.

The irradiation dose and/or tube voltage may be determined (or set) by preparing a table in which the information on the irradiation dose (mAs) and/or tube voltage (kVp) according to the relative density and/or thickness of the breast 10 is written, and then selecting in the table the irradiation dose and/or tube voltage corresponding to said information on the calculated relative density and/or the detected thickness of the breast 10.

Here, when the angle range (rₐ) is narrow, the depth of field is deep, so it is possible to take a picture at a low-resolution, a low-irradiation dose. On the contrary, when the angle range (rₐ) is wide, the rotation angle is wide, so an image may be acquired at a high-resolution and a high-irradiation dose to focus on a part of interest.

Also, the radiographic mode determination unit 140 may further comprise a resolution determination unit (not shown) to determine the resolution of the radiographic image according to at least one of the irradiation dose and the angle range (rₐ). The resolution determination unit (not shown) may determine the resolution of the radiographic image according to at least one of the irradiation dose and the angle range (rₐ), and the radiographic images may be output by controlling the resolution to be higher or lower in proportion to or inverse proportion to the optimal imaging mode and/or the optimal irradiation dose. For example, in the narrow mode, the resolution of the radiographic image may be low, and in the wide mode, the resolution of the radiographic image may be high. From the narrow mode to the wide mode, the resolution of the radiographic image may gradually increase. In this case, in the narrow mode, the angle range (rₐ) may be narrow and the irradiation dose may be low. In the wide mode, the angle range (rₐ) may be wide and the irradiation dose may be high. As described for the irradiation dose, if the angle range (rₐ) is narrow, then the resolution of the radiographic image is controlled to be lower, while if the angle range (rₐ) is wide, then the resolution of the radiographic image is controlled to be higher. Alternatively, when the angle range (rₐ) is narrow, the resolution of the radiographic image may be controlled to be higher, while when the angle range (rₐ) is wide, the resolution of the radiographic image may be controlled to be lower.

Additionally, the radiographic mode determination unit 140 may further comprise an imaging frequency determination unit (not shown) to determine the number of radiographic imaging runs according to at least one of the irradiation dose and the angle range (rₐ). The imaging frequency determination unit (not shown) may determine the number of radiographic imaging runs according to at least one of the irradiation dose and the angle range (rₐ), and the maximum number of imaging (or the number of radiographic images) may be determined in proportion to or inverse proportion to the optimal angle range (rₐ) and/or the optimal radiation dose. For example, in the narrow mode, the angle range (rₐ) is narrow, so the number of radiographic imaging runs may be reduced, leading to reducing the number of radiographic images acquired by the radiographic imaging. In the wide mode, the angle range (rₐ) is wide, so the number of radiographic imaging runs may be increased, leading to increasing the number of radiographic images acquired by the radiographic imaging.

Table 1 shows the rotation angle range (rₐ) of the radiation source for each radiographic mode on the breast according to the type of the breast, the irradiation dose to the breast, and the resolution of the radiographic image.

TABLE 1

| | Radio- | | Irradia- | |
|---|---|---|---|---|
| | graphic | Rotation angle | tion | Resolu- |
| Type of breast | mode | range | dose | tion |
| High dense type | Narrow | 7.5° | Low/ | Low/ |
| (Dense breast) | Mode | 6.5~8.5° | Medium/ | Medium/ |
| (extremely dense | | (Narrow Range) | High | High |
| 75%~) | | | | |
| Medium dense type | Little | 9.5° | Low/ | Low/ |
| (Little Dense breast) | Narrow | 8.5~10.5° | Medium/ | Medium/ |
| (heterogeneously | Mode | (Little Narrow | High | High |
| dense 51-75%) | | Range) | | |
| Scattered mammary | Normal | 12.5° | Low/ | Low/ |
| glands | Mode | 11.5~14.5° | Medium/ | Medium/ |
| (Moderate) | | (Normal Range) | High | High |
| (Normal breast) | | | | |
| (scattered | | | | |
| fibroglandular | | | | |
| 25-50%) | | | | |
| High fatty type | Wide | 25° | Low/ | Low/ |
| (Fatty breast) | Mode | 14.5~25° | Medium/ | Medium/ |
| (almost entirely | | (Wide Range) | High | High |
| fatty ~25%) | | | | |

Referring to Table 1 above, the radiation dose to the breast 10 and the resolution of the radiographic image may be selectively controlled according to the radiographic mode for the breast 10. When the rotation angle range (rₐ) of the radiation source 120 is narrow, it is possible to control the resolution of the radiographic image and the radiation dose to the breast 10 to be low.

Additionally, the radiographic imaging apparatus 100 according to the present inventive concept may further comprise a pressure control unit (not shown) configured to adjust the pressure of the compression unit 50 that presses the breast 10 according to at least one of the thickness, the plane area and the relative density information of the breast 10.

The pressure control unit (not shown) may adjust the pressure of the compression unit 50 that presses the breast 10 according to at least one of the thickness, the plane area and the relative density information of the breast 10. The pressure of the compression unit 50 may be adjusted to a pressure optimized for the type of the breast 10. That is, the pressure control unit (not shown) may control the pressure of the compression unit 50 to the optimized pressure at a level at which the subject does not feel discomfort using at least one of the calculated and/or detected relative density, thickness, and (plane) area of the breast 10. This has the effect of minimizing the discomfort of the subject (or patient) in the radiographic imaging.

For example, the optimized (compression) pressure for the identified (or calculated) type of the breast 10 may be re-calculated using compression data (or values) obtainable through the compression by the compression unit 50 after the pre-shot and at least one or a combination of two or more of the (relative) density, thickness, and (plane) area of the breast 10 obtained by the pre-shot. Thereby, the main shot (or main imaging) may be performed by maintaining the pressure of the compression unit 50 or resetting it weakly or strongly. In the type of the breast 10 with high density, the pressure may be reset slightly weakly according to the density rate. Also, the thickness of the breast 10 may be obtained through the compression (or pressing) by the compression unit 50.

The image processing unit 170 may generate a radiation image by using image information based on the detected value from the radiation detector 110. It may be a separate element from the radiation detector 110, or a part of the radiation detector 110. For example, the image processing unit 170 may acquire image information based on electrical signals from a plurality of pixel(s) read-out (or scanned) in the radiation detector 110 and generate radiographic images (e.g., the pre-shot image, the main-shot image, etc.) by performing signal processing on the image information. Also, the image processing unit 170 may reconstruct radiographic image data (or projection data) taken at a plurality of imaging angles into a three-dimensional (3D) tomography.

Additionally, a grid 161 may be provided between the breast 10 and the radiation detector 110. The grid 161 may absorb scattered radiation among the radiation that has passed through the breast 10. It may be positioned between the breast 10 and the radiation detector 110. When the radiation is irradiated to the breast 10, some of the irradiated radiation is absorbed by the breast 10, and the remaining radiation passes through the breast 10. Some of the radiation passing through the breast 10 is scattered while passing through the breast 10, and the scattered radiation may be removed by the grid 161. Only the radiation having a straightness can be detected by the radiation detector 110. That is, the grid 161 is positioned between the breast 10 and the radiation detector 110 to remove radiation scattered by the breast 10. The grid 161 can remove the scattered radiation using an incident angle difference between the main radiation straightly passing through the breast 10 and the radiation scattered through the breast 10 in a random direction.

For example, the grid 161 may include an absorption pattern (s) and a transmission pattern (s) that are alternately arranged. The absorption pattern (s) may absorb the radiation scattered through the breast 10 and block the scattered radiation from reaching the radiation detector 110. The absorption pattern (s) may be formed of metal strip (s) arranged side by side with each other. Also, the grid 161 may be a focused grid or an unfocused grid. For the focused grid, the absorption pattern (s) is inclined toward the center line of the grid 161. As the absorption pattern (s) moves away from the center line, the inclined angle of the absorption pattern (s) may increase.

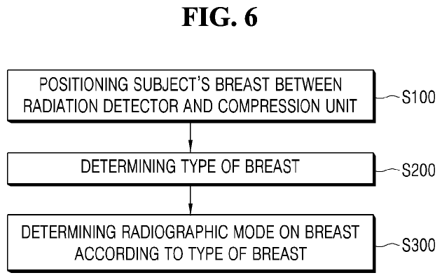

FIG. 6 is a flowchart of a radiographic imaging method according to further embodiment of the present inventive concept.

A radiographic imaging method according to further embodiment of the present inventive concept will be described in more detail with reference to FIG. 6, but matters overlapping those described above for the radiographic imaging apparatus according to an embodiment of the present inventive concept will be omitted.

According to further embodiment of the present inventive concept, a radiographic imaging method may comprise the steps of positioning a subject's breast between a compression unit and the radiation detector for radiographic imaging (S100); determining a type of the breast (S200); and determining a radiographic imaging mode for the breast according to the type of the breast (S300).

First, a subject's breast is positioned between a radiation detector a compression unit to perform the radiographic imaging (S100). The subject's breast may be positioned on the radiation detector or between the radiation detector and the compression unit to perform the radiographic imaging such as pre-shot or main shot. In this case, the subject's breast may be moved to position it on the radiation detector, or the radiation detector may be moved to position the breast thereon. The radiation detector and/or the breast (or the subject) may be moved by a transfer unit.

Next, a type of the breast is determined (S200). The type determination unit may determine the type of the breast to determine a radiographic mode suitable for the type of the breast. For example, the type of the breast may be determined using a radiation transmission value for the breast obtained by detecting radiation that has passed through the breast in the radiation detector.

Next, the radiographic mode for the breast is determined according to the type of the breast (S300). The radiographic mode determination unit may determine the radiographic mode for the breast according to the type of the breast. The rotation angle and irradiation dose of the radiation source, etc. may be determined according to type of the breast determined in the type determination unit. The radiographic mode suitable for the breast may be determined according to the type of the breast in the radiographic mode determination unit, thereby enabling to perform the radiographic imaging suitable for the type of the breast, to minimize the exposure dose to the subject, and to improve the accuracy of lesion detection. The step of determining the radiographic mode (S300) may comprise the step of determining an angle range in which the radiation source rotates around the breast according to the type of the breast (S310).

The angle range in which the radiation source rotates around the breast may be determined according to the type of the breast by the rotation angle determination unit, so micro-calcifications can be effectively detected in dense breast and masses can be effectively in fatty breast.

The rotation angle determining unit may determine a rotation angle range in which the radiation source rotates according to the type of the breast, and the angle range suitable for the breast may be determined according to the type of the breast. Thereby, minute lesions that may develop into tumors can be perfectly detected at an early stage, which can minimize the incidence of breast cancer.

The step of determining the type of the breast (S200) may determine the type of the breast by using at least one of a thickness, a plane area and a relative density information of the breast.

The type of the breast may be determined (or calculated) according to at least one or a combination of two or more of the thickness, the plane area and the relative density information of the breast in the type determination unit. For example, the relative density of the breast may be divided into a dense/slight dense/medium/fatty type; the thickness of the breast may be divided into thick/medium/thin; and the plane area of the breast may be divided into narrow/medium/wide. By combining these various types, combinational n types of the breast may be detected, and in particular, n types including the combination of dense/thick/narrow may be detected. Meanwhile, if the type of the breast 10 is the first type of dense/thick/narrow, a narrow radiographic mode may be used wherein a rotation angle range is within 6.5 to 8.5° (about 7.5°), and an irradiation dose for the narrow mode may be determined.

For example, the step of determining a type of the breast (S200) may comprise the step of acquiring the relative density information of the breast (S153) to acquire the relative density information on the breast (S2153). The relative density information of the breast may be acquired in the density information acquisition unit to determine the radiographic mode suitable for the type of the breast.

The step of determining an angle range (S310) may determine the angle range in inverse proportion to the relative density of the breast. The rotation angle determining unit may determine the angle range in inverse proportion to the relative density of the breast. Thus, in fatty breast with low density (e.g., relative density less than 25%), the radiographic imaging can be performed while rotating the radiation source within the wide rotation angle range at low-dose radiation. In high density breast (e.g., relative density greater than 75%), the radiographic imaging can be performed while rotating the radiation source within a narrow rotation angle range at relatively high radiation dose. In this case, it is possible to effectively detect masses even in fatty breast, and to effectively detect micro-calcifications in dense breast.

The radiographic imaging method may further comprise the step of performing a pre-shot on the breast (S160).

A pre-shot may be performed on the breast (S160). The radiation source may irradiate radiation to the breast positioned on the radiation detector to perform the pre-shot. In general, an irradiation dose for the pre-shot may be lower than that of the main shot.

The step of determining a type of the breast (S200) may comprise the step of acquiring the relative density information of the breast by obtaining a detection value of the radiation detector detected in the step of performing the pre-shot (S160) (S210).

The detection value of the radiation detector may be acquired during the step of performing the pre-shot (S160) to acquire the relative density information of the breast (S210). The detection value of the radiation detector may be acquired during the pre-shot, and the relative density of the breast may be calculated using the detection value. Also, a pre-shot image may be generated using the detection value of the radiation detector. Also, the step of determining a type of the breast (S200) may calculate (or acquire) the relative density of the breast using the detection value of the radiation detector obtained in the step of performing a pre-shot (S160).

The step of acquiring the relative density information of the breast (S210) may calculate the relative density of the breast according to an equation which is inversely proportional to the detection value of the radiation detector.

When the relative density of the breast is high, the radiation is largely diminished while passing through the breast, so the value obtained from the radiation detector may be low. When the relative density of the breast is low, the radiation may be less diminished while passing through the breast, so the value obtained from the radiation detector may be high. Based on this, when the value obtained from the radiation detector is low, it may be determined that the relative density of the breast is high, while when the value obtained from the radiation detector is high, it may be determined that the relative density of breast is low. Taking this into account, an equation that is inversely proportional to the values obtained from the detected radiation detector may be produced, and the relative density of the breast may be calculated using the equation.

The radiographic imaging method of the present inventive concept may further comprise the step of acquiring the size information of the breast (S150).

The size information of the breast may be acquired (S150). The size information of the breast may be acquired in the size information acquisition unit, and the size information acquired on the breast may be transmitted (transferred) to the radiographic mode determination unit. For example, the size information of the breast may be transmitted to the rotation angle determination unit and may be reflected to determine the angle range.

The step of determining an angle range (S310) may (further) reflect the size information of the breast to determine the angle range. The rotation angle determination unit may (further) reflect the size information of the breast to determine the angle range. Thereby, the radiographic imaging may be performed on the breast at the optimized rotation angle range for the type of the breast.

The step of acquiring the size information of the breast (S150) may comprise the step of measuring the thickness of the breast (S151).

The thickness of the breast may be measured (S151). The thickness of the breast may be measured in the thickness measurement unit, and the thickness information of the breast may be acquired by various methods capable of measuring the thickness of the breast.

For example, the radiographic imaging method of the present inventive concept may further comprise a step of pressing the breast (S145).

The breast may be compressed (S145). The compression unit may press the breast placed on the radiation detector. Also, the thickness of the breast may be detected (or acquired) using the compression unit. The thickness measurement unit may measure (or detect) the thickness of the breast according to a distance between the compression unit and the radiation detector. The compression unit may be consisted of only a compression panel to press the breast between the compression panel and the radiation detector, or may be consisted of a compression panel and a support panel to press the breast between the compression panel and the support panel.

The step of performing a pre-shot (S160) may be performed at an irradiation dose that is determined according to the thickness of the breast. The irradiation dose of the pre-shot may be determined according to the thickness of the breast measured in the step of measuring the thickness of the breast (S151). The diminished amount of radiation which passes through the breast may vary depending on the thickness of the breast. That is, the diminished amount of radiation may large (or increase) as the thickness of the breast increases. Thus, even when the pre-shot is performed at the same irradiation dose for all types for the breast, the radiation passing through the breast is diminished according to the thickness of the breast. Overall, the detection value of the radiation detector is affected not only the relative density of the breast but also by the thickness, so it is impossible to accurately calculate the relative density of the breast.

On the contrary, according to the present inventive concept, when the pre-shot is performed at an irradiation dose determined depending on the thickness of the breast, a difference in radiation diminishment between the relative densities of the breast at the same thicknesses can be calculated, and thus it is possible to accurately calculate the relative density of the breast.

As such, to acquire the relative density information of the breast, the pre-shot may be performed on the breast at an irradiation dose determined depending on the thickness of the breast, thereby improving the accuracy of the relative density calculation. Also, the radiographic mode can be determined according to the relative density information acquired on the breast, and the radiographic imaging can be performed according to the determined radiographic mode. Thereby, a diagnostic image of the breast with improved quality can be obtained, and various problems in the field of breast cancer diagnosis can be solved.

The step of determining an angle range (S310) may (further) reflect the thickness of the breast to determine the angle range. In the rotation angle determination unit, the angle range in which the thickness of the breast is reflected may be determined to image the entire breast using the radiation source having a fixed irradiation angle. That is, when the radiation source has a fixed irradiation angle, it may be prevented that the angle range is determined only by the relative density of the breast, thereby ensuring that the entire breast is imaged.

The step of acquiring the size information of the breast (S150) may (further) comprise the step of measuring the (plane) area of the breast (S152).

The (plane) area of the breast may be measured (S152). The area of the breast may be measured in the area measurement unit. The measured area of the breast may be transmitted to the rotation angle determination unit and be reflected to determine the angle range or to determine the irradiation angle of the radiation source.

The step of determining an angle range (S310) may (further) reflect the (plane) area of the breast to determine the angle range. If the angle range is determined without considering the (plane) area of the breast 10, the radiographic imaging may be performed on the breast having large (plane) area using the narrow rotation angle range, or the radiographic imaging may be performed on the breast having small (plane) area using the wide rotation angle range. In this case, a mass that may be on the outer side of the breast may be missed, or a micro-calcification cluster that may be on the outer side of the breast may be missed. To avoid this problem, the rotation angle determination unit may determine the angle range in which the (plane) area of the breast is reflected. The angle range may be proportional to the (plane) area of the breast.

The radiographic imaging method of the present inventive concept may further comprise the step of generating a radiographic image using the detected value from the radiation detector obtained in the step of performing a pre-shot (S160) (S170).

A radiographic image may be generated using the detected value from the radiation detector obtained (or detected) in the step of performing a pre-shot (S160) (S170). The radiographic image may be generated using the detected value from the radiation detector obtained in the step of performing a pre-shot (S160) in the image processing unit, and a pre-shot image may be acquired during the step of performing a pre-shot (S160).

The step of measuring the (plane) area of the breast (S152) may calculate the area of the breast using a radiographic image generated in the step of generating a radiographic image (S170). The outline of the breast may be identified (or acquired) along the light and dark distribution of the radiographic image (i.e., the pre-shot image) generated in the step of generating a radiographic image (S170). The area of the breast can be calculated as the area of the breast within the radiographic image generated in the step of generating a radiographic image (S170) by using a ratio of the area of the radiographic image generated in the step of generating a radiographic image (S170) and the detection area of the radiation detector.

The area of the breast within the radiographic image generated in the step of generating a radiographic image (S170) may be calculated (or obtained) by the number of pixels. For example, if a ratio of the area of the radiographic image generated in the step of generating a radiographic image (S170) and the detection area of the radiation detector is 1:10, the area of the breast 10 can be calculated by multiplying the area of the breast within the radiographic image generated in the step of generating a radiographic image (S170) by 10 times.

That is, the radiographic imaging method of the present inventive concept may further comprise the step of measuring the area of the breast using the radiographic image generated in the step of generating a radiographic image (S170) (S152).

According to the present inventive concept, the radiographic mode (e.g., the rotation angle range of the radiation source) may be determined according to the type of the breast that is identified using at least one of the thickness, the (plane) area and the relative density information of the breast, and thus the radiographic imaging may be performed on the breast in the optimized radiographic mode such as the optimized rotation angle range of the radiation source for the type of the breast. Thus, defects (or noises) such as cutting artifacts generated during image reconstruction for a three-dimensional (3D) tomosynthesis image can be minimized. As a result, the lesion detectability and accuracy can be maximized and the reliability of medical examination and diagnosis can be improved.

Also, in the step of determining an angle range (S310), the rotation angle determination unit may determine the angle range by applying a weight to the relative density, thickness, and area of the breast. Since the relative density and thickness of the breast are inversely proportional to the angle range, and the plane area of the breast is proportional to the angle range, if a wight is not applied to the relative density, thickness and area of the breast, the rotation angle range may be determined to be uncertain such as a normal range or a slight narrow range regardless of the relative density of the breast. As a result, it is impossible to perform the radiographic imaging suitable for the type of the breast, as well as there may be problems that the exposure dose to the breast is excessively increased, or it is difficult to accurately detect lesions using the produced (or acquired) radiographic images. Thus, a weight may be applied to the relative density, thickness, and area of the breast, and the rotation angle determination unit may determine the rotation angle range with the weight applied to the relative density, thickness, and area of the breast.

For example, the largest weight may be applied to the relative density of the breast, and the thickness and area of the breast may be weighted according to the overall size of the breast.

The step of determining a radiographic imaging mode (S300) may further comprise the step of determining an irradiation dose to the breast according to at least one of the thickness, the plane area and the relative density information of the breast (S320).

The irradiation dose to the breast may be determined according to at least one of the thickness, the plane area and the relative density information of the breast (S320). The irradiation dose to the breast may be determined according to at least one of the thickness, the plane area and the relative density information of the breast in the irradiation dose determining unit 142, and the irradiation dose of the main shot may be determined. The irradiation dose determination unit may determine the irradiation dose to the breast in proportion to the thickness of the breast as well as the relative density of the breast.

For example, the irradiation dose and/or tube voltage may be determined (or set) by preparing a table in which the information on the irradiation dose (mAs) and/or tube voltage (kVp) according to the relative density and/or thickness of the breast is written, and then selecting in the table the irradiation dose and/or tube voltage corresponding to said information on the calculated relative density and/or the detected thickness of the breast.

When the angle range is narrow, the depth of field is deep, so it is possible to take a picture at a low-resolution, a low-irradiation dose. When the angle range is wide, the rotation angle is wide, so an image may be acquired at a high-resolution and a high-irradiation dose to focus on a part of interest.

The step of determining a radiographic imaging mode (S300) may further comprise the step of determining the resolution of the radiographic image according to at least one of the irradiation dose and the angle range (S330).

The resolution of the radiographic image may be determined according to at least one of the irradiation dose and the angle range (S330). The resolution of the radiographic image may be determined according to at least one of the irradiation dose and the angle range in the radiographic mode determination unit, and the radiographic images may be output by controlling the resolution to be higher or lower in proportion to or inverse proportion to the optimal imaging mode and/or the optimal irradiation dose. For example, in the narrow mode, the resolution of the radiographic image may be low, and in the wide mode, the resolution of the radiographic image may be high. From the narrow mode to the wide mode, the resolution of the radiographic image may gradually increase. In this case, in the narrow mode, the angle range may be narrow and the irradiation dose may be low. In the wide mode, the angle range may be wide and the irradiation dose may be high. As previously described for the irradiation dose, if the angle range is narrow, then the resolution of the radiographic image is controlled to be lower, while if the angle range is wide, then the resolution of the radiographic image is controlled to be higher. Alternatively, when the angle range is narrow, the resolution of the radiographic image may be controlled to be higher, while when the angle range is wide, the resolution of the radiographic image may be controlled to be lower.

The step of determining a radiographic imaging mode (S300) may further comprise the step of determining the number of radiographic imaging runs according to at least one of the irradiation dose and the angle range (S340).

In addition, the number of radiographic imaging runs may be determined according to at least one of the irradiation dose and the angle range (S340). The number of radiographic imaging runs may be determined according to at least one of the irradiation dose and the angle range in the radiographic mode determination unit 140 and the maximum number of imaging (or the number of radiographic images) may be determined in proportion to or inverse proportion to the optimal angle range and/or the optimal radiation dose. For example, in the narrow mode, the angle range is narrow, so the number of radiographic imaging runs may be reduced, leading to reducing the number of radiographic images acquired by the radiographic imaging. In the wide mode, the angle range is wide, so the number of radiographic imaging runs may be increased, leading to increasing the number of radiographic images acquired by the radiographic imaging.

As described above, according to the present inventive concept, the type a subject's breast can be determined in the type determination unit and a radiographic mode such as an imaging angle can be suitably determined according to the type of the breast, so that the accuracy of lesion detection can be improved while minimizing the exposure dose to the subject. Thus, minute lesions that may develop into tumors can be perfectly detected at an early stage, which has an effect of minimizing the incidence of breast cancer. Also, by determining an angle range in which a radiation source rotates around the breast according to the type of the breast, it is possible to effectively detect micro-calcifications in dense breast, and also masses in fatty breast. To acquire the relative density information on the breast, a pre-shot is performed on the breast at an irradiation dose determined according to the thickness of the breast. Thus, the accuracy of the relative density calculation can be improved, as well as the relative density information thus acquired can be used to determine a radiographic mode such as an angle range in which a radiation source rotates and to perform the radiographic imaging, resulting in a high-quality diagnostic image on the breast. Also, there is an effect that can solve various problems in the field of breast cancer diagnosis. Additionally, to determine a radiographic mode, the radiographic mode can be determined according to the type of the breast which is identified using at least one of the thickness, the plane area and the relative density information of the breast and the radiographic imaging can be performed in the radiographic mode such as an angle range in which a radiation source rotates which is optimized for the type of the breast, so defects such as cutting artifacts generated during image reconstruction for a three-dimensional (3D) tomosynthesis image can be minimized. As a result, the lesion detectability and accuracy can be maximized and the reliability of medical examination and diagnosis can be improved.

Although the preferred embodiments of the present inventive concept have been illustrated and described above, the present inventive concept is not limited to these embodiments, and those of ordinary skill in the art to which the present inventive concept pertains will understand that various modifications and equivalent other embodiments may be implemented therefrom without departing from the scope of the present inventive concept as claimed in the claims. Accordingly, the technical protection scope of the present inventive concept should be defined by the following claims.

1. A radiographic imaging apparatus comprising:
a radiation source configured to irradiate radiation to a subject's breast and to be rotatable around the breast;
a radiation detector configured to detect radiation that has passed through the breast;
a type determination unit configured to determine a type of the breast using a radiation transmission value for the breast obtained from the radiation detector; and
an imaging mode determination unit configured to determine a radiographic mode for the breast according to the type of the breast.

2. The radiographic imaging apparatus according to claim 1, wherein the imaging mode determination unit comprises a rotation angle determination unit configured to determine an angle range in which the radiation source rotates according to the type of the breast.

3. The radiographic imaging apparatus according to claim 2,
wherein the type determination unit comprise at least one of:
a thickness measurement unit configured to measure a thickness of the breast;
an area measurement unit configured to measure a plane area of the breast; and
a density information acquisition unit configured to acquire a relative density information on the breast, and
determines the type of the breast using at least one of the thickness, the plane area and the relative density information of the breast.

4. The radiographic imaging apparatus according to claim 3, wherein the rotation angle determination unit determines the angle range in inverse proportion to a relative density of the breast.

5. The radiographic imaging apparatus according to claim 3, wherein the density information acquisition unit acquires the relative density information on the breast using the radiation transmission value obtained by a pre-shot.

6. The radiographic imaging apparatus according to claim 5, wherein a relative density of the breast is inversely proportional to the radiation transmission value.

7. The radiographic imaging apparatus according to claim 5, wherein the pre-shot is performed at an irradiation dose determined according to the thickness of the breast.

8. The radiographic imaging apparatus according to claim 3, wherein the imaging mode determination unit further comprises an irradiation dose determination unit configured to determine an irradiation dose to the breast according to at least one of the thickness, the plane area and the relative density information of the breast.

9. The radiographic imaging apparatus according to claim 8, wherein the imaging mode determination unit further comprises a resolution determination unit configured to determine a radiographic image resolution according to at least one of the irradiation dose and the angle range.

10. The radiographic imaging apparatus according to claim 8, wherein the imaging mode determination unit further comprises an imaging frequency determination unit configured to determine the number of radiographic imaging runs according to at least one of the irradiation dose and the angle range.

11. A radiographic imaging method comprising the steps of:
positioning a subject's breast between a radiation detector and a compression unit, for radiography;
determining a type of the breast; and
determining a radiographic mode for the breast according to the type of the breast.

12. The radiographic imaging method according to claim 11, wherein the determining the radiographic mode comprises determining an angle range in which a radiation source rotates around the breast according to the type of the breast.

13. The radiographic imaging method according to claim 12, wherein the determining the type of the breast determines the type of the breast using at least one of a thickness, a plane area and a relative density information of the breast.

14. The radiographic imaging method according to claim 13, wherein the determining the angle range determines the angle range in inverse proportion to a relative density of the breast.

15. The radiographic imaging method according to claim 13, further comprising the steps of:
performing a pre-shot on the breast, and
wherein the determining the type of the breast comprises acquiring the relative density information of the breast by obtaining a detection value of the radiation detector detected in the performing the pre-shot.

16. The radiographic imaging method according to claim 15, wherein the performing the pre-shot is performed at an irradiation dose determined according to the thickness of the breast.

17. The radiographic imaging method according to claim 15, wherein the acquiring the relative density information of the breast calculates a relative density of the breast according to an equation inversely proportional to the detection value of the radiation detector.

18. The radiographic imaging method according to claim 13, wherein the determining the radiographic mode further comprises determining an irradiation dose to the breast according to at least one of the thickness, the plane area and the relative density information of the breast.

19. The radiographic imaging method according to claim 18, wherein the determining the radiographic mode further comprises determining a radiographic image resolution according to at least one of the irradiation dose and the angle range.

20. The radiographic imaging method according to claim 18, wherein the determining the radiographic mode further comprises determining the number of radiographic imaging runs according to at least one of the irradiation dose and the angle range.
